(19) United States
(12) Patent Application Publication
(10) Pub. No.: US 2007/0016131 A1
(43) Pub. Date: Jan. 18, 2007

(54) Flexible magnets for navigable medical devices

(76) Inventors: Gareth T. Munger, St. Louis, MO (US); Michael E. Sabo, St. Louis, MO (US); Rogers C. Ritter, Charlottesville, VA (US)

Correspondence Address: HARNESS, DICKEY, & PIERCE, P.L.C, 7700 BONHOMME, STE 400, ST. LOUIS, MO, 63105, US

(21) Appl. No.: 11/314,805

(22) Filed: Dec. 21, 2005

Related U.S. Application Data
(60) Provisional application No. 60/698,540, filed on Jul. 12, 2005.

(51) Int. Cl. A61M 31/00; A61M 25/00

(52) U.S. Cl. 604/95.05; 600/585; 604/95.01

(57) ABSTRACT

A magnetically navigable catheter or guide wire having a proximal and a distal end, and a magnetically responsive structure that surrounds at least a portion of the catheter or guide wire at the distal end, wherein the magnetically responsive structure is comprised of a flexible magnetically responsive material.

### CROSS-REFERENCE TO RELATED APPLICATIONS

This application claims the benefit of U.S. provisional application Ser. No. 60/637,505, filed Dec. 20, 2004, and U.S. provisional application Ser. No. 60/698,540, filed Jul. 12, 2005, the entire disclosures of which are herein incorporated by reference.

### FIELD OF THE INVENTION

This invention relates to magnetically navigable catheters and guide wires, and more particularly to magnetically navigable catheters and guide wires having a flexible magnetically responsive element.

### BACKGROUND OF THE INVENTION

Medical catheters and guide wires are typically used for delivering medical devices to target locations within the vasculature of the body. Navigation of a conventional guide wire involves rotating or applying a torque to the proximal end of the guide wire repeatedly to rotate the distal tip while the wire is pushed. This action is repeated until, by trial and error, the tip enters the desired vessel branch. After the guide wire has made several bends, the guide wire may become increasingly difficult to control, requiring repeated attempts to enter a desired vessel branch or gain access through an occlusion. This trial and error method can frustrate the physician and cause additional wall contact and potential trauma.

Magnetically navigable guide wires have been developed with magnetically responsive elements near the distal end which can be controlled through the application of a magnetic field external to the patient. An example of a magnetically navigable guide wire is disclosed in Magnetically Navigable Guide wire, U.S. patent application Ser. No. 10/337/326, filed Jan. 7, 2003, published as US 2003-0127571 A1 on Jul. 10, 2003 and incorporated herein by reference. When the distal end of the guide wire is adjacent the vessel of interest, the user operates a magnetic system to apply a magnetic field to deflect the guide wire tip to align generally with the opening of a vessel side branch. The magnet system can often direct the distal end of the guide wire into the branch on the first effort, eliminating the trial and error of manually operated guide wires and thereby reducing or eliminating trauma to the vessel wall. Additional potential benefits derived from magnetic navigation include reduction in intervention time and decrease in patient and medical personnel exposure to x-ray radiation dose.

Medical catheters have also been provided with a magnetically responsive element by which the distal end of the catheter can be navigated, or oriented by the application of a magnetic field. An example of a magnetically navigable catheter is disclosed in Werp et al., U.S. Pat. No. 5,931,818, for Method Of And Apparatus For Intraparenchymal Positioning Of Medical Devices, incorporated herein by reference. Catheters must be flexible enough for the tip to be significantly deflected in response to an applied magnetic field in order to gain access to small vessels, while also being strong enough to resist kinking that can arise when trying to navigate tight spaces and small vessels within a vasculature system. However, two competing considerations apply to the design of magnetically navigable catheters and guide wires: minimizing the use of rigid materials to maintain flexibility while providing a sufficient amount of magnetically responsive material for enabling magnetic navigation of the distal end.

Various magnetic surgery systems have been developed to create a magnetic field in a selected direction in an operating region of a subject's body to orient a magnetic medical device in the body, such as those disclosed in U.S. Pat. No. 6,241,671, issued Jun. 5, 2001, for Open Field System for Magnetic Surgery, and U.S. Pat. No. 6,015,414, issued Jan. 18, 2000, for Method and Apparatus for Magnetically Controlling Motion Direction of a Mechanically Pushed Catheter, the disclosures of which are incorporated herein by reference.

### SUMMARY OF THE INVENTION

The present invention relates to a magnetically navigable catheter or guide wire having a proximal and a distal end, and a magnetically responsive structure that surrounds at least a portion of the catheter or guide wire at the distal end, wherein the magnetically responsive structure comprises a flexible magnetically responsive material. The magnetically navigable catheter or guide wire having a flexible magnetically responsive material has a distal tip that is capable of being deflected a minimum angle, when subjected to a magnetic field having a known magnitude and orientation. The total magnetic responsiveness of a magnetic layer or structure is called the “magnetic moment.” In a permanent magnet this moment is the product of the effective internal magnetization (per unit volume) times the volume, or more generally is given by the volume integral of the elemental effective internal magnetization. In a magnet of permeable material this moment will depend on the external field that is present to magnetize (usually to a partial degree) its volume.

Previous magnetically navigable guide wires and catheters have typically used permanent magnet tips, preferably of the strongest (permanent) magnetic material Neodymium Iron Boron (NeFeB), which is very stiff and brittle. These tips are often 2 mm long or longer, and are rigidly fixed to the distal end of the wire or catheter. This stiff tip, although small in length, may still be significant compared to the blood vessel diameter in many cases, and therefore it is difficult for the tip to make sharp turns in such vessels. An advantage of the present invention over previous magnetically navigable catheters and guide wires is that the magnetic guiding element, being of a flexible material, can be longer overall, but bendable with a shorter turning radius than that of the previous devices. The inventors have found that the flexible tipped catheters and guide wires of the present invention are capable of negotiating sharper turns in smaller vessels than the previous magnetically navigable versions of these devices.

In accordance with one aspect of the present invention, a medical device such as a guide wire is provided that comprises an elongate wire having a proximal and a distal end, and a flexible magnetically responsive structure surrounding a portion of the elongate wire adjacent the distal end. The magnetically responsive structure is comprised of such material and of sufficient size to substantially orient the distal end of the elongate wire relative to an externally applied magnetic field. In the preferred embodiment of this aspect of the invention, the flexible magnetically responsive structure comprises a wound coil of flexible magnetic wire surrounding the distal end portion of the elongate wire.

In accordance with another aspect of the present invention, a medical device such as catheter is provided that comprises a tubular member having a proximal and a distal end, a lumen therebetween, and a magnetically responsive structure that surrounds at least a portion of the tubular member at the distal end, wherein the magnetically responsive structure element is comprised of a flexible magnetically responsive material. In the preferred embodiment of this aspect of the present invention, the flexible magnetically responsive structure comprises a wound coil of flexible magnetic wire surrounding the distal end portion of the catheter. In another embodiment of the present invention, the flexible magnetically responsive structure comprises a braided sheath of flexible magnetic wire surrounding the distal end portion of the catheter.

At least some embodiments of the medical devices of this invention are adapted to be introduced into the body through an artery of the patient's vasculature, and can be deflected up to at least 30° in any direction upon the application of a magnetic field of no more than 0.1 Tesla, and more preferably no more than about 0.08 Tesla. The medical device is preferably sufficiently stiff to allow it to be mechanically advanced in the selected direction.

### BRIEF DESCRIPTION OF THE DRAWINGS

FIG. 1 is a side elevation view of a catheter in accordance with the principles of the present invention;

FIG. 2 is a graph illustrating the magnetic parameters of a preferred flexible magnetically responsive material in the present invention;

FIG. 3 is a side elevation view of a guide wire in accordance with the principles of the present invention; and

FIG. 4 is a side elevation view of another embodiment of a guide wire in accordance with the principles of the present invention.

Corresponding reference numerals indicate corresponding parts throughout the several views of the drawings.

### DETAILED DESCRIPTION OF THE INVENTION

The magnetic material used in the preferred embodiment of this invention is a reasonably magnetically strong permanent magnet, and yet not be brittle (as most permanent magnets are) so that it can be made into a bendable, conformable structure at or near the tip of a guide wire or catheter. A spring-like coil or a braid is one geometrical configuration that is appropriate to the medical uses intended. NdFeB, the magnetically strongest permanent magnet material, is brittle, and not flexible enough for use as a bendable coil or braided sheath. Samarium Cobalt is another magnetically strong permanent magnet material that, although mechanically stronger than NdFeB, is unlikely to be useful as a flexible spring. Platinum Cobalt, a permanent magnet alloy, is more ductile (although still hard), and a good candidate for the material of this invention. Platinum Iron is another such alloy that might be used.

The Platinum Cobalt (PtCo) material typically possesses a residual induction (remaining magnetization level Bᵣ of a permanent magnet when removed from the magnetizer) that is lower than desired for application in magnetically guided devices that use NdFeB, and it is therefore not as magnetically strong. In addition, the “coercive force” H_{c} of PtCo materials are lower than that of NdFeB, and therefore PtCo is more vulnerable to incidental demagnetization. [The incidental demagnetization can occur in a number of ways. In a permanently magnetized material parallel aligned domains repel each other, are intrinsically unstable, and are held in place by a “coercive force”. In effect the coercive force is a mechanical tendency for the material to resist any tiny geometrical changes that would allow the otherwise securely aligned domain boundaries (Bloch Boundaries) to develop a permanent shift to reorganize their shapes and effectiveness in response to the ultra minute mechanical warping. At elevated temperatures thermal agitation can result in minute changes in the material structure which allow the domains to reorient to some extent, causing a temporary or permanent loss of magnetization. Similarly, when a strong external magnetic field is applied such as in the range used for navigation, it can also result in such rearrangements.] However, the Platinum Cobalt material, when subjected to heat-treatment typically with parameters of 1000° Celsius for 3 hours and quenching at 600° Celsius for 10 hours, yields a material having a relatively high H_{c}. And, important for the present application, this somewhat hard alloy is much less brittle than NdFeB, which consists of compressed, aligned and sintered grains. Thus, PtCo can have properties necessary for its use in the present invention. It will be apparent to those skilled in the art that other materials with favorable properties suitable for the purposes of this invention might be identified upon studies of modified heat treatments similar to those described therein.

The Platinum Cobalt alloy possesses a coercivity H_{c} of about 5 to 6 KOe, and an energy product BHₘₐₓ of about 8 MGOe. FIG. 2 shows a Hysteresis graph of B, the induction 42, vs. H the applied field 40. The Bᵣ, or remnant magnetism 44 is roughly the residual magnetic field that remains within the material after the magnitude of an applied magnetizing field has been reduced to zero. For appropriate geometries, and with magnetization in a direction parallel to the axis of a wound coil, this is a good indicator of the degree of magnetization of the material in that coil, and of its magnetic responsiveness. The H_{c}, or coercive field 46 is the field that is required to subsequently reduce the residual magnetization to zero. As stated above, this measures the ability of the material to avoid reduction of its magnetic moment in the presence of magnetically disturbing elements. A product B×H can be formed from any rectangle that touches the BH curve in the second quadrant, and has a base and side on the axes as shown in 48. The largest area of such B×H rectangles in the second quadrant, BHₘₐₓ, is a standard measure of the intrinsic 4“magnetic strength” of the material. It has the units of energy, and is called the maximum energy product.

Recent advances in work with Platinum-Iron have led to significant iimprovement of this material. It has generally been known to have a significantly higher Br (magnetic field retentivity after magnetization) than Platinum Cobalt, and only lacked good coercivity. This involves the addition of Niobium (Nb) to the alloy, so that it is a Fe—Pt—Nb system. The magnetic advance was actually shown in 1991 (Kiyoshi Watanabe, in Materials Transactions JIM, Vol. 32, No. 3 (1991), pp 292 to 298.) In this article 60 kinds of Fe—Pt—Nb alloys were homogenized by heating at high temperatures, quenching in ice-water and then tempering at 723-1023 K. The typical Fe-39.5Pt-0.75Nb alloy exhibited a Br of 1.05 T and coercivity Hc of about 5 kOe.

A large number of experiments, primarily aimed at forming Fe—Pt material, useful at temperatures up to 150 C, have considerably increased the Hc of this and other more complicated versions of this material. Hiroshi Yamamoto and Ryuki Monma (Digest No BS 11 in IEEE International Magnetics Conference “Intermag Asia 2005) have made ribbons of Pr—Fe—Co—Ti—B—Si systems with some remarkable properties. One such material exhibited Hc˜197 kOe, but with Br reduced. Another version of these ribbons exhibited Hc˜69 kOe with Br˜0.8 T.

In practice a permanent magnetic material is usually preferable for navigation. In such a material, the maximum magnetic strength achievable (through a prior magnetization procedure) is not dependent upon the application of a magnetic field concurrent with navigation. The magnetic components of prior magnetically navigable catheter and medical guide wire devices have consisted of permanent magnet materials such as NdFeB. While Hiperco, a material with magnetic permeability and some degree of ductility, has been utilized in some of these medical devices, its magnetization is induced by the navigating field and the effective magnetic response of this material is significantly less than that of other good permanent magnetically responsive materials. A further limitation of Hiperco is that a level of induced magnetization comparable to that of many permanent magnet materials is achieved only at fields of magnitudes well above those used in magnetic navigation.

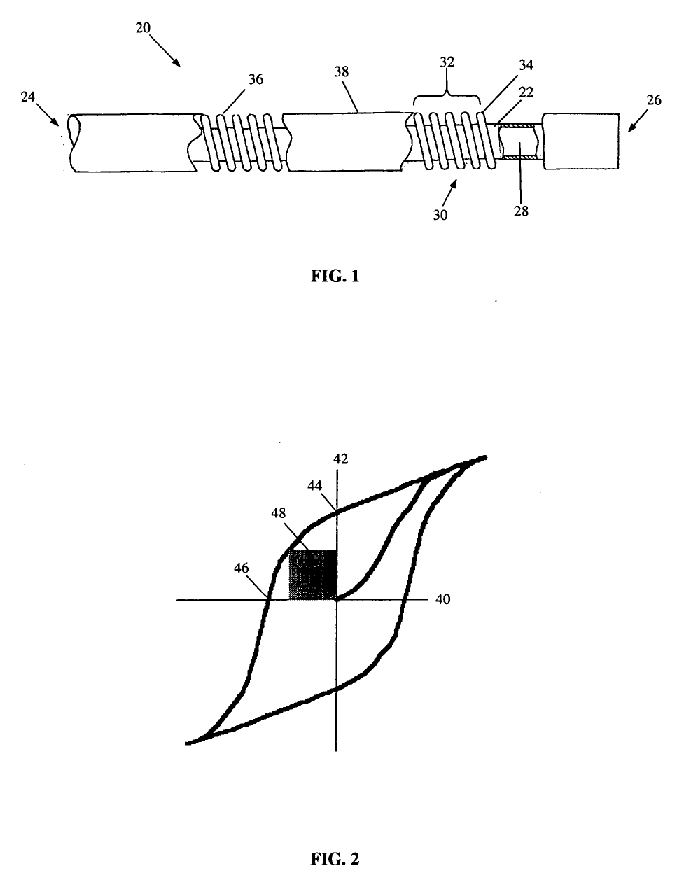

One embodiment of a magnetically navigable medical catheter device in accordance with the principles of the present invention is indicated generally as 20 in FIG. 1. The magnetically navigable catheter device 20 comprises a tubular member 22 having a proximal end 24, a distal end 26, a lumen 28 there between, and a magnetically responsive structure 30 that surrounds at least a portion of the tubular member 22 at or near the distal end 26. The magnetically responsive structure 30 comprises a flexible magnetically responsive material. The flexible magnetically responsive structure may comprise a wound coil 32 of flexible magnetically responsive wire 34 surrounding the distal end portion of the elongate tubular member 22, or in an alternate construction, a braided sheath of flexible magnetically responsive wire surrounding the distal end portion of the elongate tubular member 22.

The magnetically responsive material in either the wound coil 32 or the braided sheath may comprise a flexible permeable material or a flexible permanent magnetic material. As described above, the stiffness of NdFeB material used in previous magnetically navigable medical devices mitigates against its use the present flexible tipped devices. Platinum Cobalt is an alloy under the name Platinex, manufactured by General Electric. When processed appropriately it exhibits a balance of flexibility and permanent magnetization suitable for use in the present invention. Another material Platinum Cobalt Chromium alloy might have similar properties. And as described above, Fe—Pt—Nb and other alloys are “hard magnetically” while not being as brittle as ceramic NdFeB. A further advantage of several of these magnetic platinum alloys is that they usually have a high fraction of platinum and therefore are inherently quite radiopaque, facilitating imaging of the device with conventional x-ray imaging systems.

The relevance of demagnetization in a permanent magnet material is that it can reduce the responsiveness of the material. This can occur initially from unfavorable geometric shapes of the material. Elongated cylinders are most favorable. In addition this can arise for several other elements acting on limitations in the material. One of these limitations is the loss of some degree of magnetization of the material by aging. Another limitation is the resistance to loss of magnetization by application of an external field in a direction not along the original magnetization axis. For embodiments of this invention which use a coiled wire of permanent material the responsiveness of the element suffers relative to that of cylinders in that it is an unfavorable geometry for the maximum development of initial magnetization in the magnetizing process, and will additionally be less favorable in resisting demagnetization by the applied navigating field. The magnetizing field, and the intended device magnetization are essentially along the coil axis. The individual turns of the coil are approximately orthogonal to this direction, so the magnetization is predominantly across the thin wire of material. Elements of pitch, and of wire diameter relevant to coil diameter affect the magnitude of this effect. Thus an initially lower magnetization in conjunction with a vulnerability to reduction by external field can act against the desired effectiveness of this embodiment (and others) of this invention. The resistance of the material to demagnetization is called the “coerciveness, or coercivity” and is measured by the H_{c} 46 in the diagram of FIG. 2 and as described above.

Platinum Cobalt material, when subjected to the heat-treatment parameters of 1000° Celsius for 3 hours and quenching at 600° Celsius for 10 hours, yields a material having such desirable magnetic characteristics. One embodiment of a medical device produced with this material according to the principles of the present invention possesses a significant flexibility and a coercivity sufficiently high to avoid major demagnetization in typical navigating magnetic fields of at least 0.06 Tesla and more preferably at least 0.08 Tesla. The coercivity is preferably such that the material retains at least about 70 percent of its magnetization in an applied navigation field. Alternatively, the coercivity is preferably such that the device can still bend 30° over a distance of 10 mm, in a applied field of no more than 0.08 Tesla, and more preferably in an applied field of no more than 0.06 Tesla. As stated above, the inventors have found that when processed this way the Platinum Cobalt alloy possesses the magnetic parameters of a coercivity H_{c} of about 5 to 6 KOe, and an energy product BHₘₐₓ of about 8 MGOe. With a flexible magnetically responsive structure comprising a PtCo coiled wire having a wire diameter in the range 0.001 to 0.006, and preferably 0.002 inch to 0.004 inch, The distal end 26 of the catheter medical device 20 can be bent at a 4 mm radius of curvature, and more preferably a 3 mm radius of curvature, without permanently kinking. The catheter medical device 20 of the present invention may further comprise a second wound coil 36 made of a stainless steel or other permeable material, the second wound coil being disposed proximally adjacent to the Platinum Cobalt wound coil 32. The catheter medical device 20 may further comprise an outer coating 38 made of a hydrophilic material, or the coating 38 may alternatively comprise a polymeric material encapsulating the magnetically responsive coil 32 and the stainless steel wound coil 36.

The preferred embodiment of the medical catheter device 20 may include a layer of radio opaque material disposed around a portion of the coiled wire on the distal end of the catheter, where the radiopaque material enables viewing of the medical guide wire in an X-ray Fluoroscopic Imaging system. An example of such a radiopaque material is platinum or a platinum alloy. In the preferred embodiment, the magnetic material itself is sufficiently radiopaque. It should be noted that other materials exhibiting a balance of flexibility and magnetic properties may be also be used to suitably obtain similar parameters of flexibility and magnetic response of the catheter medical device in accordance with the principles of the present invention.

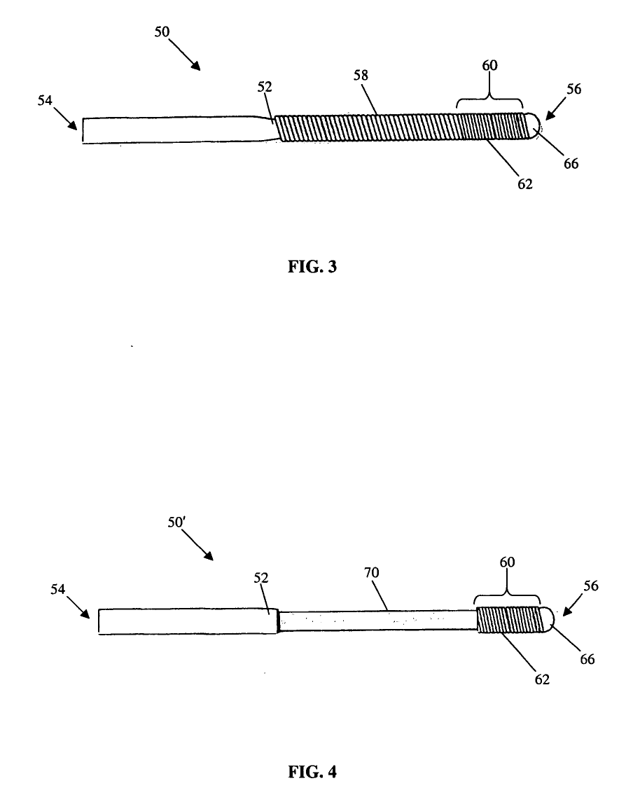

In another aspect of the present invention, a medical guide wire 50 shown in FIG. 3 is provided that comprises an elongate wire 52 having a proximal end 54 and a distal end 56, and a flexible magnetically responsive structure 60 surrounding a portion of the elongate wire adjacent the distal end 56. The flexible magnetically responsive structure 60 comprises a wound coil 62 of flexible magnetically responsive wire surrounding the distal end portion of the elongate wire 52, where the magnetically responsive wire 62 is of such material and sufficient size to substantially align the distal end 56 of the elongate wire relative to an externally applied magnetic field. The wound coil of flexible magnetically responsive wire 62 also possesses sufficient flexibility to allow the distal tip to bend at a 4 mm radius of curvature, and more preferably at a 3 mm radius of curvature, without permanently kinking. In one preferred embodiment of this aspect of the invention, the flexible magnetically responsive material comprises a Platinum Cobalt alloy having an H_{c} of about 5 to 6 KOe, and a BHₘₐₓ of about 8 MGOe. In another preferred embodiment of this aspect of the invention, the flexible magnetically responsive material comprises an Iron Platinum alloy having and Hc of more than 10 Koe and a Br of more than 1.0 T. (It is to be understood that other elements may be added to the Iron and Platinum in order to achieve better magnetica and mechanical performance.) The guide wire 50 may further include a second wound coil 58 of magnetically permeable material proximal to the Platinum Cobalt wound coil 62, where the second wound coil 58 provides structural support to the guide wire 50. This structural support is useful in allowing the guide wire to progress through occlusions and in navigating through highly curved vessel anatomy. In some embodiments in which even greater radio opacity is desired, beyond that supplied by the PtCo wire, or if an alternate magnetic material is used, the distal end of the medical guide wire 50 may further include a layer of radiopaque material of sufficient density to enable viewing of the medical guide wire in an X-ray Fluoroscopic Imaging system.

The distal end of the medical guide wire 50 further comprises a rounded tip element 66 secured to the end of the elongate wire 52. The rounded tip element 66 may be brazed or welded to the end of the elongate wire 52, and preferably comprises a ball or oval shape. The rounded tip element 66 is preferably made of stainless steel or Hiperco, but in other embodiments, it may also be made of a magnetically permanent material such as Platinum Cobalt, which would be both magnetic and radiopaque. The magnetically responsive rounded tip element 66 and the flexible magnetically responsive layer 60 would both serve to substantially align the distal end of the elongate wire 52 relative to an externally applied magnetic field.

In yet another embodiment of the present invention shown in FIG. 4, the magnetically responsive layer 60 of the medical guide wire 50′ may comprise a polymer shaft 70 in place of the wound coil 58 of permeable magnetic material. Some embodiments of a medical guide wire may further comprise an optional hydrophilic coating (not shown) over the distal tip of the guide wire.

The parameters for the magnetically responsive material of several embodiments of a medical catheter or guide wire device in accordance with the principles of the present invention are such that the tip of a medical device is capable of being deflected a minimum amount when subjected to an applied magnetic field. The maximum deflection of the distal tip can be determined by holding the wire at a set distance proximal to the tip such as 0.5 inches, and applying a magnetic field of known magnitude, H, at varying angles to the tip until the maximum tip deflection is observed. For example, in the Stereotaxis Niobe™ magnetic navigation system, a field of 0.1 Tesla can be applied within the subject in any direction. The maximum deflection angle of a medical device in a 0.1 Tesla field is thus one way to characterize the medical device performance in the Niobe™ magnetic navigation system. The inventors have determined that the tip of the medical device in accordance with the principles of the present invention is capable of being deflected a minimum of 30 degrees relative to the orientation of the distal end of the medical device, when subjected to a magnetic field having a magnitude of not more than 0.1 Tesla and more preferably not more than 0.08 Tesla and even more preferably not more than 0.06 Tesla. The applied magnetic field in this example has a reference angle of about 90° degrees relative to the longitudinal axis of the distal end of the medical device.

The advantages of the above-described embodiment and improvements should be readily apparent to one skilled in the art, as to enabling magnetically navigation of a flexible catheter or guide wire medical device. Other examples of medical devices that may incorporate the above improvements include Electrophysiology catheters, flexible endoscopes, electrodes for ablation, balloon or stent delivery catheters and surgical tools. Additional design considerations may be incorporated without departing from the spirit and scope of the invention. Accordingly, it is not intended that the invention be limited by the particular embodiment or form described above, but by the appended claims.

What is claimed is:

1. A medical device for use in a subject's body, the medical device comprising:
an elongate member having a proximal end and a distal end;
a flexible magnetically responsive structure that surrounds at least a portion of the elongate member adjacent the distal end, the flexible magnetically responsive structure allowing the portion of the elongate member it surrounds to bend at a radius of about 4 mm without permanent deformation of the flexible magnetically responsive structure.

2. The medical device according to claim 1 wherein the flexible magnetically responsive structure is capable of causing the elongate member to bend at least 30° over a distance of 10 mm in response to an applied field of no more than 0.08 Tesla.

3. The medical device according to claim 2 wherein the flexible magnetically responsive structure is capable of causing the elongate member to bend at least 30° over a distance of 10 mm in response to an applied field of no more than 0.06 Tesla.

4. The medical device according to claim 1 wherein the flexible magnetically responsive material has a sufficient coercivity to retain at least 70% of its magnetization in an applied field of at least 0.08 Tesla.

5. The medical device according to claim 1 wherein the flexible magnetically responsive material has a sufficient coercivity to retain sufficient magnetization in applied field of at least 0.08 Tesla to bend at least 30° over a distance of 10 mm in response to the applied field.

6. The medical device of claim 1, wherein the flexible magnetically responsive structure comprises a coil of flexible magnetically responsive wire surrounding the distal end portion of the elongate member.

7. The medical device of claim 6, wherein the flexible magnetically responsive wire and the stiffness of the distal end portion of the elongate member are such that the tip of the medical device is capable of being deflected a minimum of 30 degrees over a length of 1 cm, when subjected to a magnetic field having a magnitude of 0.08 Tesla.

8. The medical device of claim 1 wherein the coil is magnetized generally parallel to the axis of the coil.

9. The medical device of claim 1, wherein the flexible magnetically responsive structure comprises a braided sheath of flexible magnetically responsive wire surrounding the distal end portion of the elongate member.

10. The medical device according to claim 1 wherein the flexible magnetically responsive wire comprises a flexible permanent magnetic material.

11. The medical device according to claim 10, wherein the flexible permanent magnetic material is made of a platinum cobalt alloy.

12. The medical device according to claim 11, wherein the flexible permanent magnetic material is made of a platinum nickel cobalt alloy.

13. The medical device according to claim 10 wherein the permanent magnetic material is made of a platinum iron alloy.

14. The medical device according to claim 13 in which minor amounts of other elements are added to the material.

15. The medical device according to claim 1 wherein the flexible magnetically responsive wire comprises a flexible permeable magnetic material.

16. The medical device according to claim 1, wherein the magnetic material has an H_{c} greater than 4 KOe, and wherein the magnetic material has an energy product greater than 6 MGOe

17. The medical device according to claim 16, wherein the magnetic material has an H_{c} greater than 6 KOe, and wherein the magnet material has an energy product greater than 8 MGOe

18. The medical device according to claim 6, further comprising a layer of radiopaque material disposed around a portion of the coiled wire on the distal end of the elongate member forming the tip of the catheter.

19. The medical device according to claim 18, wherein the radiopaque material comprises a platinum alloy.

20. The guide wire of claim 1 wherein the magnetically responsive structure is radiopaque.

21. The guide wire of claim 20 where the flexible magnetically responsive structure is plated with a radiopaque material.

22. The medical device according to claim 1 wherein the medical device is a catheter, and wherein the elongate member has a lumen therein.

23. The medical device according to claim 1 wherein the medical device is a guide wire, and wherein the elongate member is a flexible wire.

24. The guide wire according to claim 23 wherein the flexibility of the distal end portion of the elongate wire is greater than that of the proximal portion of the elongate wire.

25. The guide wire of claim 23, wherein the more flexible portion at the distal end of the elongate wire comprises one or more tapered sections that reduce in wire cross-section.
